(19) United States
(12) Patent Application Publication
(10) Pub. No.: US 2022/0227839 A1
(43) Pub. Date: Jul. 21, 2022

(54) COLLAGEN NANOPARTICLES FROM SCALY FISH SKIN

(71) Applicants: Ezgi BOZ, Nilüfer, Bursa (TR); BAHÇESEHIR ÜNIVERSITESI, Istanbul (TR)

(72) Inventors: Ezgi BOZ, Nilüfer, Bursa (TR); Fatih ÖZDENER, Istanbul (TR)

(21) Appl. No.: 17/609,529

(22) PCT Filed: Jul. 22, 2020
(86) PCT No.: PCT/TR2020/050645

§ 371 Date: Nov. 8, 2021

(30) Foreign Application Priority Data
Jul. 24, 2019 (TR) 2019/11143

(51) Int. Cl. C07K 14/78; A61K 8/65; A61K 8/02; A61Q 19/00; A61K 9/51; A61L 15/32; A23L 33/17; A23J 1/00; A23J 1/04; A23J 1/10; A23K 20/147

(52) U.S. Cl. CPC C07K 14/78; A61K 8/65; A61K 8/0216; A61Q 19/00; A61K 9/5169; A61L 15/325; A23V 2002/00; A23J 1/002; A23J 1/04; A23J 1/10; A23K 20/147; A61K 2800/10; A61K 2800/413; A23L 33/17

(57) ABSTRACT

Disclosed are collagen nanoparticles obtained from waste scaly skins from fish by using a micro fluid technique, a method for obtaining the collagen nanoparticles, and their field of use.

### TECHNICAL FIELD

The invention, is related to the collagen nanoparticles obtained from the waste scaly skins from the fish by using micro fluid technique; to the method for obtaining said collagen nanoparticles and their field of use.

### STATE OF THE ART

Today, it is well known that collagen is a structural protein which forms the soft tissues such as dermis, ligament, tendons, vessels and hard tissues such as bones. Collagen constitutes 6% of body weight of the mammals and 25-30% of all body proteins. Collagen is the protein which is most commonly found in the soft and solid tissues of the body such as tendon, ligament, skin, cornea, cartilage, bone, blood vessels, gout, namely gastrointestinal system, teeth, gums, vertebrae, discs (vertebrae), heart valves etc. The biodegradable collagen may be used for treatment surfaces in medicine. The collagen is usually used in various types of drug administration, vector systems for controlled release of active substance emulsions, pastes, eye washes, and vector systems for various containers. It is known that collagen is a physiological modulator in tissue repair as a structure matrix protein. There is a relationship between collagen and cells (blood platelets, lymphocytes, and macrophages), growth factors, cytokines, other matrix proteins (for example fibronectin and hyaluronic acid) and coagulation factors. Collagen is known to have an apparent debriding effect, reduces the number of bacteria and increases the epithelisation area in superficial wounds. It was also seen that it activates the inflammatory phagocytic cells and increases the vascularity of the repair tissue.

The amino acid mostly found in the structure of the collagen is Glycine. One glycine repeats every 3 amino acids in the collagen chain. Although there are approximately 20 known collagen types, the most famous ones among them are Type 1 and Type 2. While Type 1 collagen forms skin, tendon, ligament and bones, Type 2 collagen forms the cartilage tissue. The following applications are encountered in the literature regarding the subject matter:

The patent document numbered TR 2010/09159 describes a collagen-based and biologically absorbable dressing and thus; this dressing contains a determined proportion of linear polymer biguanide and/or a salt of this substance which is soluble in water. As biguanide the polyhexamethylenebiguanide is preferred.

Another invention is the patent document numbered KR0837858 and describes a method for preparing water-soluble oligopeptides from pork skin collagen by means of irradiation.

Accordingly the method comprises the following process steps; dissolving the pork skin collagen in 0.05-0.1 M NaCI in an amount of 5 to 10 times (W/V) in 50 to 300 kGy doses and adding 0.5-1% papain enzyme to the water soluble collagen, allowing the mixture to wait for 0.5 to 4 hours and cross-sectioning the enzyme-processed substance into specific oligopeptides.

It is known that collagen from mammals to have excellent biocompatibility, however when it is used in medical applications, there is a risk of animal disease from collagen from mammals to the subject. This situation has caused focusing on marine species as they are plenty of both in number and variety. Moreover, it is known that costs are less in obtaining collagen from marine species. In the first studies made for achieving collagen from marine species, collagen was obtained by hydrolysis from waste fish scales and films suitable for use as a plaster were produced from the same. In another study, sponge structured collagens were obtained from the salmon fish and it was investigated whether they can be used as artificial tissue or not. In another study, the collagen sponges obtained from fish scales were added to the extracts obtained from Macrotyloma uniflorum plant and a hybrid structure was obtained. It has been found that this hybrid structure is resistant to collagenase enzyme, biocompatible and antibacterial and it can be used as a material that can be used to cover wound/burn surfaces. In a recent study, the collagen-apatite composites from the collagens obtained from the scaly fish are prepared in different ratios by means of microwave and the effect of this composite on tooth tissue regeneration is investigated. It is observed that the composite containing 40% collagen gives the best result in vessel formation. While the sea collagen can be administered orally, it cannot be injected, for this reason its effect is limited to the creams and masks used.

All studies mentioned above were not only insufficient in creating a desired small sized collagen but also were insufficient in forming a system from the fish skin for accelerating the blood clotting. The fields of use of the collagen are limited since the required size of collagen cannot be obtained.

As a result due to the abovementioned disadvantages and the insufficiency of the current solutions regarding the subject matter, a development is required to be made in the relevant technical field.

### BRIEF DESCRIPTION OF THE INVENTION

The present invention is related to collagen nanoparticles obtained from the scaly skins of the fish which fulfils the abovementioned requirements, eliminates all disadvantages and brings some additional advantages.

The main aim of the invention is to provide a method in which the micro fluid technique is used for obtaining collagen nano particles from the scaly fish skins and to provide collagen nanoparticles produced with this method.

An aim of the invention is to provide a collagen product with high absorption feature and with a wide range of use since the obtained collagen is nano-sized and fish origin. It is found that the collagen obtained from the fish skins can be absorbed 1.5 times more by the human body and can be mixed with the blood faster than the collagen obtained from other sources. In this respect, it is mentioned that the collagen obtained from the fish skin is more suitable for the medical applications.

An aim of the invention is to provide a biocompatible collagen product to be used in human and veterinary medical products by means of the antimicrobial properties of collagen nanoparticles from the sclay fish which both stop bleeding and accelerate healing.

An aim of the invention is to provide an environmentally friendly and economical production method in which the bio-wastes can be evaluated as the raw material together with obtaining collagen nanoparticles from the waste scaly fish skins.

In order to fulfil the abovementioned aims mentioned above, the invention is a method for obtaining collagen nanoparticles from the scaly fish skins by using micro fluid technique, comprises the following process steps; boiling the scaly fish skins in a solvent; filtering the liquid collagen extract obtained by boiling;obtaining collagen micro bubbles by simultaneously sending the filtered liquid collagen extract and an inert gas to the micro channels in a micro fluid device; The invention also covers the collagen nanoparticles obtained by said method and the use of said collagen nanoparticles. The structural and characteristic features of the present invention will be understood clearly by the following drawings and the detailed description made with reference to these drawings and therefore the evaluation shall be made by taking these figures and the detailed description into consideration.

### FIGURES CLARIFYING THE INVENTION

FIG. 1 is the tension-strain curve of the dried form the extract obtained from the scaly fish skin and water.

FIG. 2 is the tension-strain curve of the dried form the extract obtained from the scaly fish skin and vinegar.

FIG. 3a, is the view of the haemostasis experiment results of the blood to which serum physiological is added as the control group.

FIG. 3b, is the view of the haemostasis experiment results of the blood to which undiluted fish skin fluid extract is added as the experimental group.

FIG. 4a, is the view of the haemostasis experiment results of the blood to which serum physiological is added as the control group.

FIG. 4b, is the view of the haemostasis experiment results of the blood to which fish skin fluid extract diluted in a ratio of 1/10is added as the experimental group.

FIG. 5 is the SEM image of collagen micro bubbles.

FIG. 6a is the SEM view of the collagen nanoparticles of the invention with 5000-magnification.

FIG. 6b is the SEM view of the collagen nanoparticles of the invention with 30000-magnification.

FIG. 6c is the SEM view of the collagen nanoparticles of the invention with 60000-magnification.

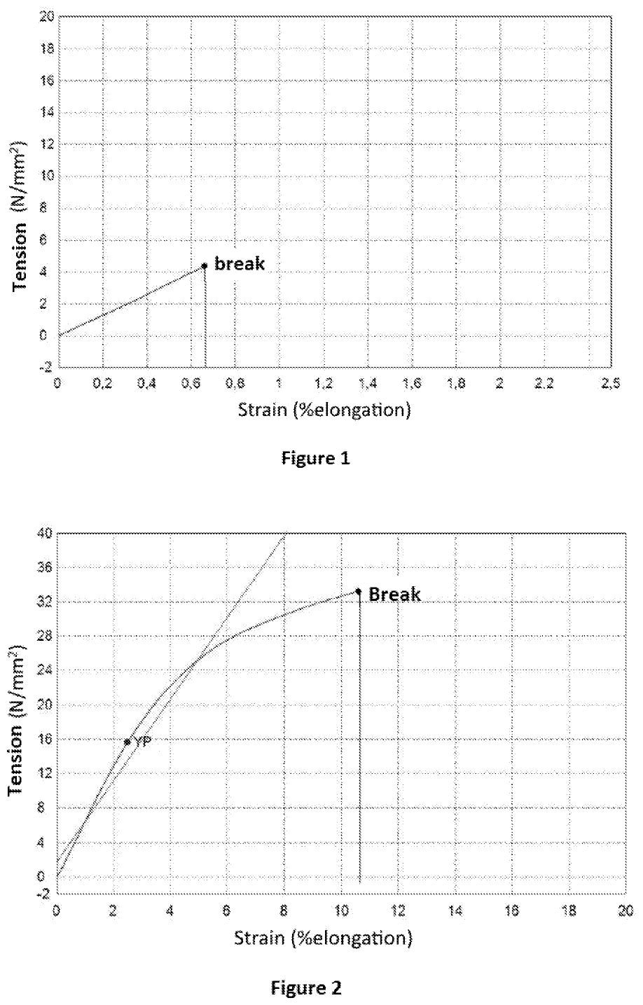

### DETAILED DESCRIPTION OF THE INVENTION

In this detailed description, the inventive method is described only for clarifying the subject matter in a manner such that no limiting effect is created.

The invention, is related to the collagen nanoparticles obtained from the waste scaly skins from the fish by using micro fluid technique; to the method for production of said collagen nanoparticles and their field of use.

The method in which the micro fluid technique is used for obtaining collagen nano particles from the scaly fish skins mainly comprises the following process steps;

i. Boiling the scaly fish skins in a solvent,

ii. Filtering the liquid collagen extract obtained as a result of boiling process,

iii. Obtaining collagen micro bubbles by means of sending the filtered liquid collagen extract and an inert gas simultaneously to the micro channels within the micro fluid device,

iv. Obtaining collagen nano particles by means of drying the collagen micro bubbles.

In one embodiment of the invention; in the process step (i) preferably; scaly fish skin and the solvent in a ratio of 1:1 by weight: volume are boiled at a temperature of 100-135° C. for 5-10 minutes.

In an embodiment of the invention, the scaly fish skin is the waste scaly fish skin that is remained from the fish production. Thus a waste product is converetd into a product with high added value.

In one embodiment of the invention, as said scaly fish skin, an individual or combinations selected from the following group is used; carp skin, salmon skin, gray mullet skin, sea bass skin, coral fish skin, sea bream skin, dentex skin and/or gilthead seabream skin. In a preferred embodiment of the invention, the scaly skins obtained from the carp fish are used.

In one embodiment of the invention said solvent is selected from the following group individually or in combinations; distilled water, isotonic water, serum physiological, alkaline water, vinegar, tap water and/or acetic acid. In a preferred embodiment of the invention, distilled water is used as the solvent.

Collagen nanoparticles are obtained for the first time using the micro fluid technique for the first time by means of the developed invention. According to an embodiment of the invention, the inert gas which is preferred in the micro fluid device is the nitrogen gas.

Different inert gases can be used in the alternative applications. Moreover, the obtained collagen nanoparticles are obtained in cubic form in a shape controlled manner and this type of collagen structure is developed for the first time.

In the studies made by the owners of the invention, a study of an example of the invention is submitted herein below in detail.

For the example study, 100 grams of scaly fish skin was taken from the carp fish and was boiled in 100 ml distilled water at 100° C. temperature for approximately 10 minutes. The liquid extract that contains obtained fish collagen was filtered through a mesh filter and the solid materials were removed. In one channel of a micro fluid device having two channels with T shape, the filtered liquid extract is input and in the other channel the inert nitrogen gas is input. The micro bubbles formed as a result of the operation of the device were dropped onto a glass surface and thus dried at 21° C. temperature for 3 days. As a result of drying the micro bubbles; collagen nanoparticle with cubic form were obtained. The obtained collagen nanoparticles were detected by means of the scanning electron microscope (SEM).

In the abovementioned study; the proteins contained in the filtered liquid collagen extract were analysed and the results are given herein in the Table 1 below.

TABLE 1
Proteins that are contained in the liquid extract

| | | | | | | Normalized | Raw | Spectral |
|---|---|---|---|---|---|---|---|---|
| | | | | | | abundance | abundance | counts |
| | Number of | Unique | Safety | | | Condition 1 | Condition 1 | Condition 1 |
| Participation | peptides | peptides | point | Mass | Description | MC_170219 | MC_170219 | MC_170219 |
| O34194 | 1 | 1 | 6.1485 | 44257.8 | 60 kDa chaperonin | 32403.76 | 32403.76 | 1 |
| | | | | | (Protein Cpn60) | | | |
| | | | | | (groEL protein) [symbol] | | | |
| P02452; P02453; | 1 | 1 | 6.1502 | 139910.1 | Collagen alpha 1(I) | 77903.39 | 77903.39 | 1 |
| P02457; Q9XSJ7 | | | | | chain precursor. | | | |
| P02563; P02564; | | | | | | | | |
| P12883; P13533; | | | | | | | | |
| P13539; P13540; | | | | | | | | |
| P79293; Q02566 | | | | | | | | |
| P11087 | 1 | 1 | 4.8174 | 224307.1 | Myosin heavy | 1100.048 | 1100.048 | 1 |
| | | | | | chain_cardiac | | | |
| | | | | | muscle alpha | | | |
| | | | | | isoform ( [symbol] | | | |
| P17756 | 2 | 2 | 5.5321 | 138970 | Collagen alpha 1(I) | 17558.9 | 17558.9 | 1 |
| | | | | | chain precursor [symbol] | | | |
| P31431 | 1 | 1 | 8.5997 | 58850.15 | GAG polyprotein | 25068.39 | 25068.39 | 2 |
| | | | | | [Contains: Core | | | |
| | | | | | protein P16; Core [symbol] | | | |
| P96471 | 1 | 1 | 5.0724 | 21641.6 | Syndecan-4 precursor | 3979.466 | 3979.466 | 1 |
| | | | | | (Amphiglycan) (SYND4) | | | |
| | | | | | (Ryudoc [symbol] | | | |
| Q57766 | 1 | 1 | 11.9468 | 49910.21 | Streptokinase precursor [symbol] | 522727.3 | 522727.3 | 4 |
| | | | 11.8248 | 32786.84 | Formylmethanofuran-- | 148898.1 | 148898.1 | 2 |
| | | | | | tetrahydromethanopterin | | | |
| | | | | | formyl [symbol] | | | |

[symbol]

indicates data missing or illegible when filed

In the abovementioned study; the cytotoxicity tests of the filtered liquid collagen extract were performed and the results are given herein in the Table 2 below.

They are the measurements taken at 450/630 according to the WST1 viability test. Three 100 ul was taken from each well and the transferred ones into 96-well plate were measured. 3 plastic controls (cells which grow completely in their own conditions and there is nothing on them), 3 controls with whatman (cells which grow in the conditions wherein the whatman paper is placed on the cells) and 5 cells which grow in the conditions where extract impregnated whatman paper is placed on the cells. The experiment is made with L929 cells.

TABLE 2
Cytotoxicity Test Results

| | | | | | | | | Std |
|---|---|---|---|---|---|---|---|---|
| | | | | | average | | Average | deviation |
| Plastic Control 1 | Plastic cells 1 | 3.523 | 3.670 | 3.636 | 3.610 | 100 | 100 | 0.679 |
| Plastic Control 2 | Plastic cells 2 | 3.571 | 3.694 | 3.705 | 3.657 | 101 | | |
| Plastic Control 3 | Plastic cells 3 | 3.553 | 3.703 | 3681 | 3.646 | 101 | | |
| Watman Control 1 | PBS Cells with watman 1 | 3.544 | 3.653 | 3.662 | 3.620 | 100 | 100 | 0.328 |
| Watman Control 2 | Empty Cells with watman 2 | 3.519 | 3.638 | 3.631 | 3.596 | 99 | | |
| Watman Control 3 | Empty Cells with watman 3 | 3.549 | 3.634 | 3.636 | 3.606 | 100 | | |
| Experiment 1 | Cells added extract 1 | 3.602 | 3.674 | 3.666 | 3.647 | 101 | 101 | 0.775 |
| Experiment 2 | Cells added extract 2 | 3.695 | 3.700 | 3.717 | 3.704 | 102 | | |
| Experiment 3 | Cells added extract 3 | 3.697 | 3.696 | 3.625 | 3.673 | 101 | | |
| Experiment 4 | Cells added extract 4 | 3.682 | 3.691 | 3.716 | 3.696 | 102 | | |
| Experiment 5 | Cells added extract 5 | 3.650 | 3.631 | 3.644 | 3.642 | 101 | | |

In said study; the filtered liquid collagen extract was analysed by the ASTM E2149 Antibacterial Test Method. The results are given in the following table. It is stated in A: liquid collagen extract obtained from the scaly fish skin and water; B: liquid collagen extract obtained from the scaly fish skin + vinegar.

TABLE 3
Antibacterial efficacy values against Staphylococcus aureus
(ATCC 6538)^{a} according to ASTM 2149 Test Method

| | | 30 minutes | | 2 hours | | 5 hours | |
|---|---|---|---|---|---|---|---|
| Item | | Reduction in bacteria | | | | | |
| number | Sample | (%) | log | (%) | Log | (%) | log |
| 1 | A | 3.00 | 0.01 | −6.50 | −0.03 | −19.50 | −0.09 |
| 2 | B | −100.00 | −4.66 | −100.00 | −4.66 | −100.00 | −4.66 |

^{a}The bacteria concentration transferred to each sample with 1 gram weight is calculated as 4.54 × 10⁴ (log 4.66) cfu*/ml.
*cfu: Colony forming unit.
Note:
(+) shows the % bacteria values representing the increase in the number of bacteria, (−) shows the % bacteria value representing the decrease in the number of bacteria. (−) 100 value indicates all bacteria on the surface.

TABLE 4
Antibacterial efficacy values against Escherichia coli
(ATCC 35218)^{b} according to ASTM 2149 Test Method

| | | 30 minutes | | 2 hours | | 5 hours | |
|---|---|---|---|---|---|---|---|
| Item | | Reduction in bacteria | | | | | |
| number | Sample | (%) | log | (%) | Log | (%) | log |
| 1 | A | 12.50 | 0.05 | 43.75 | 0.16 | 127.08 | 0.36 |
| 2 | B | 2.08 | 0.01 | 21.88 | 0.09 | 31.25 | 0.12 |

^{b}The bacteria concentration transferred to each sample with 1 gram weight is calculated as 2.08 × 10⁴ (log 4.34) cfu*/ml.
*cfu: Colony forming unit.
Note:
(+) shows the % bacteria values representing the increase in the number of bacteria, (−) shows the % bacteria value representing the decrease in the number of bacteria. (−) 100 value indicates all bacteria on the surface.

In said study; the filtered liquid collagen extract was dropped onto a glass surface in a thin film and thus dried at 21° C. temperature for 3 days. The steam permeability of the dried collagen extract film was analysed and the results are given herein Table 5 in the following.

TABLE 5
The Steam Permeability of the Dried Conditions of
the Extracts Obtained from the Scaly Fish Skin

| | 21.6° 42% | 30° 34% | 40° 27% |
|---|---|---|---|
| | MOISTURE | MOISTURE | MOISTURE |
| Empty | 79.31537 | 406.6933 | 496.0771 |
| Parafilm | 0.514756 | 0.782228 | 1.695479 |
| Fish Skin + Water | 24.66541 | 29.00761 | 50.66489 |
| Fish Skin + Vinegar | 18.12371 | 24.64018 | 48.57048 |

In the abovementioned study; the mechanical tests were performed by recording the tension and strain values of the samples prepared in 4 cmx 1 cm width/length dimensions from the dried collagen extract film obtained by drying the filtered liquid collagen extract under a pulling force having 5 %/min increase rate. In Figure the tension-strain curve of the dried form the extract obtained from the scaly fish skin and water is shown. In FIG. 2 the tension-strain curve of the dried form the extract obtained from the scaly fish skin and vinegar is shown.

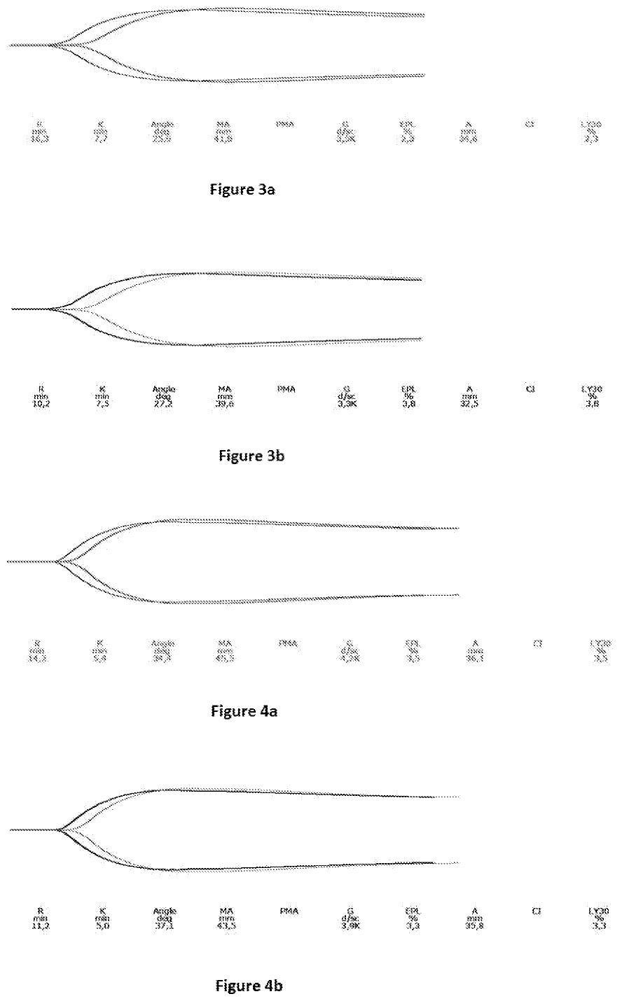

In said study; the haemostasis experiment is made to the filtered liquid collagen extract. The experiment results of the blood to which undiluted fish skin fluid extract is added as the experimental group and the blood to which serum physiological is added as the control group are given in FIG. 3a and FIG. 3b. The experiment results of the blood to which undiluted fish skin fluid extract is added in a ratio of 1/10as the experimental group and the blood to which serum physiological is added as the control group are given in FIG. 4a and FIG. 4b.

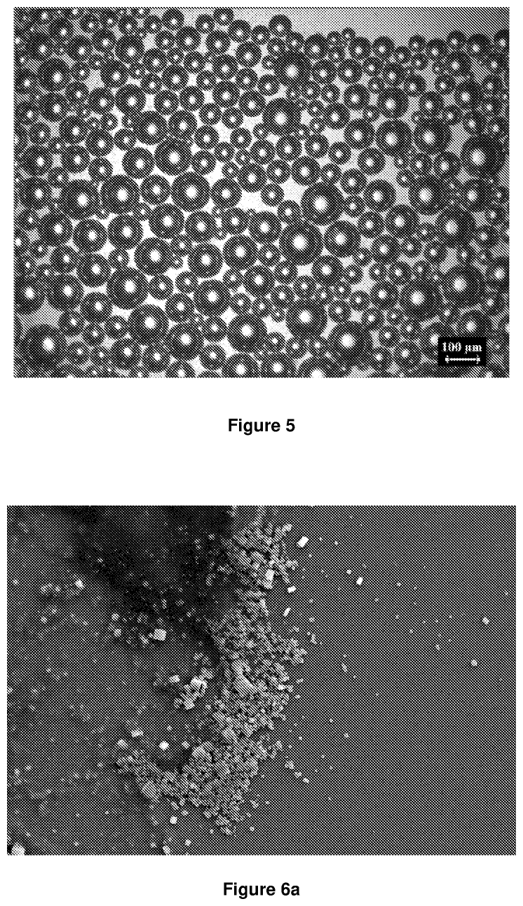

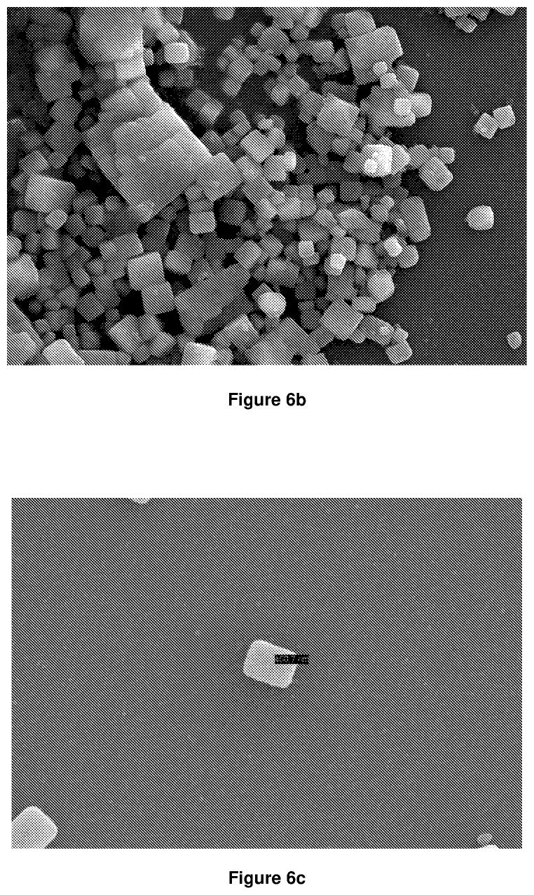

In said study; the collagen nanoparticles obtained by the micro fluid technique were detected by means of the scanning electron microscope (SEM). In FIG. 5 the SEM image of collagen micro bubbles obtained from the micro fluid device is given. In FIG. 6, SEM image of the collagen nanoparticles in cubic form obtained by means of drying the collagen micro bubbles is given (FIG. 6-a 5000 magnification, FIG. 6b 30000 magnification, FIG. 6-c 60000 magnification).

In the invention it is provided that the collagen obtained from the fish skins can be absorbed 1.5 times more by the human body and can be mixed with the blood faster than the collagen obtained from other sources. This condition leads to an advantage in terms of medical uses. Moreover, the fish skin facilitates the production step and reduces the cost. Since it is a biological material, the side effects caused by chemical substances do not occur. The invention covers the use of said nanoparticles obtained from the scaly fish skin as an auxiliary substance in human and veterinary medical products. An example of this type of use of an appropriate plaster or dersssing containing said collagen nanoparticles for use in skin wounds and bleedings. Also said collagen nanoparticles can be used as an anti-bleeding, healing accelerator, antimicrobial or cell regenerating agent. The invention also covers the use of the nanoparticles obtained from said scaly fish skin as a food supplemet or feed supplement. The examples for this type of uses are the beverages containing collagen nanoparticles or capsullescontaining collagen nanoparticles. The invention also covers the use of the nanoparticles obtained from said scaly fish skin in dermathological and cosmetic products. The cosmetic forms such as base, gel, cream, serum or injections for the emulsions and cosmetic creams containing the inventive collagen nanoparticles are examples to this type of use. The invention also covers the use of the nanoparticles obtained from said scaly fish skin in the textile products. During the production of fabrics that contact with the skin, the required textile products can be obtained by using the encapsulated method as an anti-bleeding, healing accelerator, antimicrobial or cell regenerating agent.

1. A method in which the a micro fluid technique is used for obtaining collagen nano particles from scaly fish skins, characterized in that; it comprises the following process steps:
i. boiling the scaly fish skins in a solvent,
ii. filtering liquid collagen extract obtained as a result of the boiling process,
iii. obtaining collagen micro bubbles by means of sending the filtered liquid collagen extract and an inert gas simultaneously to micro channels within a micro fluid device, and
iv. obtaining collagen nano particles by means of drying the collagen micro bubbles.

2. The method according to claim 1, wherein said scaly fish skins comprise waste scaly skins obtained from fish.

3. The method according to claim 1, wherein said solvent is distilled water, isotonic water, serum physiological, alkaline water, vinegar, tap water and/or acetic acid.

4. The method according to claim 3, wherein said solvent is distilled water.

5. The method according to claim 1, wherein said scaly fish skin is carp skin, salmon skin, grey mullet skin, sea bass skin, coral fish skin, sea bream skin, dentex skin and/or gilthead seabream skin.

6. The method according to claim 5, wherein said scaly fish skin is a carp skin.

7. The method according to claim 1, wherein said inert gas is nitrogen gas.

8. The method according to claim 1, wherein in the process step of (i) a 1:1 ratio of scaly fish skin by weight:volume and the solvent are boiled.

9. The method according to claim 1, wherein in the process step of (i) the boiling process is realized during 5-10 minutes at 100-135° C. temperature.

10. Collagen nanoparticles obtained from the scaly fish skins with the method according to claim 1.

11. The use of the nanoparticles according to claim 10 as an auxiliarry substance in human and veterinary medical products.

12. The use of the collagen nanoparticles according to claim 10 as a food supplement or feed supplement.

13. The use of the collagen nanoparticles according to claim 10 in dermatological or cosmetic products.

14. The use of the collagen nanoparticles according to claim 10 in textile products.

15. Plaster or dressing comprising collagen nanoparticles according to claim 10.
